(19) United States
(12) Patent Application Publication
(10) Pub. No.: US 2010/0121367 A1
(43) Pub. Date: May 13, 2010

(54) Device for dissecting and cutting in endoscopic surgery

(75) Inventors: Yun-Nan Lin, Kaohsiung City (TW); Lin Hsu Li-Mei, Kaohsiung City (TW)

Correspondence Address: BACON & THOMAS, PLLC, 625 SLATERS LANE, FOURTH FLOOR, ALEXANDRIA, VA, 22314-1176, US

(73) Assignee: YUN-NAN LIN, Kaohsiung City (TW)

(21) Appl. No.: 12/314,537

(22) Filed: Dec. 12, 2008

(30) Foreign Application Priority Data
Nov. 13, 2008 (TW) 097143792

(51) Int. Cl. A61B 17/3201

(52) U.S. Cl. 606/174

(57) ABSTRACT

A device for tissue dissecting and cutting in endoscope surgery which primarily comprises a holding part, a transmitting part and a cutting part. The holding part offers stable support for the surgeon to perform operation without incurring sway. The transmitting part is a straight force delivery system that can deliver the force straight and forward from the holding part to the cutting part. By using this device, the surgeon can deliver the exact force for the endoscope surgery.

### FIELD OF THE INVENTION

The present invention relates to a device for dissecting and cutting soft tissue including the internal organs inside the abdominal cavity and thoracic cavity during an endoscopic surgery. As such mechanism complies with normal mechanics and exerts force stably, operator can adapt to the operation under the environment of an endoscopic surgery so as to accurately dissect and cut tissue in the surgery.

### BACKGROUND OF THE INVENTION

Conventional scissor structure used in endoscopic surgery, as shown in FIG. 1, includes a finger grip 101 for exerting force, a fixed finger grip 102 for supporting when exerting force, in which the top end of the finger grip 101 has a gear-like structure 103 serving to drive the spindle in the cavity to rotate when exerting force; a cavity 112 serving as a force output conversion space when exerting force, in which a spindle 104 and a stationary rod 105 are mutually connected and housed in the cavity 112 to serve as a force output member when exerting force, the front end of the stationary rod 105 has a slot 106, in which a sliding pin 109 therein serves as a connection point for the stationary rod 105 and a two-piece sheet body 110 having a left-and-right grip 111 and as a stopper for opening and closing the two-piece sheet body 110, the stationary rod 105 is loosely sleeved thereon by a bushing 107, a slot 108 is opened on the front end of the bushing 107 to provide the operational space for opening and closing the left-and-right grip 111, and the two-piece sheet body is operated to cut a targeting object.

During the operation, please refer to FIG. 1 and FIG. 2-2. By exerting force forwardly through the finger grip 101 and fixing with the fixed finger grip 102, force is transmitted from the gear-like structure 103 over the top end of the finger grip 101 to the spindle 104 in the cavity 112; the sliding pin 109 in the slot 106 on the front end of the stationary rod 113 is driven to move forward through the spindle 104 and to push the two-piece sheet body 110 forward; in collaboration with the bushing 107 loosely sleeved on the stationary rod 105 and the left-and-right grip 111 in the slot 108 located on the front end of the bushing 107, the opening operation of the two-piece sheet body 110 to the left and right is thus completed.

When closing the two-piece sheet body 110, please refer to FIG. 1 and FIG. 2-1 for the operation. By exerting force to pull backward through the finger grip 101 and fixing with the fixed finger grip 102, force is transmitted from the gear-like structure 103 over the top end of the finger grip 101 to the spindle 104 in the cavity 112; the sliding pin 109 in the slot 106 at the front end of the stationary rod 113 is driven to retract backward; in collaboration with the sliding pin 109 pushing the two-piece sheet body 110 and the bushing 107 loosely sleeved on the stationary rod 105 and moving backward, the slot 108 at the front end of the bushing 107 is retracted backward and the sliding pin 108 in the slot 108 departs from the two-piece sheet body 110 accordingly such that the left-and-right grip 11 is then pressed into the bushing 107 in completion of the closing operation of the two-piece sheet body 110.

In fact, conventional skill usually pertains to an erected grip. When applying force to the finger grip 101, force is transmitted to the spindle 104 via the force output member in the cavity, the spindle 104 and the stationary rod 105, and the gear-like structure 103 to further pull or push the bushing 107 so as to reach the point of force application in the end; consequently, the two-piece sheet body 110 can then finish the operation of cutting a targeting object. Because the point of force application and the finger grip 101, the fixed finger grip 102 and the point of strength, and the sliding pin 109 and the two-piece sheet body 110 are all perpendicular and force fails to be directly transmitted to the point of strength through the spindle 104 and the bushing 107, as a result, the magnitude of force on the point of strength is hard to be controlled and the resulting improper magnitude of force will be harmful to tissue. Furthermore, major movement in operation originates from the force application by thumb while the rest of fingers serve to fix and assist force application. When thumb exerts force, the rest of fingers may exert force out of instinct as well. Therefore, it will result in sway of the main body 100, leading to an unstable situation; meanwhile, it is difficult to aim at smaller target to dissect and cut, making the prior art less precise during operation.

To sum up, all instrument used in endoscopic surgery take the form of force application differing from regular surgery instrument and whose force is indirectly transmitted through relevant mechanism and magnitude of force on the point of strength is barely controllable. Such surgical operation makes the endoscopic surgery that simply requires technical demand even harder.

As far as the precision of surgery and operational simplicity and convenience is concerned, a new device for dissecting and cutting in endoscopic surgery is required indeed to resolve various problems arising from prior arts.

### SUMMARY OF THE INVENTION

In accordance with a first aspect of the present invention, a device for dissecting and cutting soft tissue, including internal organs inside abdominal cavity and thoracic cavity, and subcutaneous muscle, nerves, and blood vessels of limbs, is provided. For sake of being highly stable, uneasy to sway and operable, the device can accurately hold a targeting object. A standard horizontal grip, i.e. the prevailing holding style, is adopted, such that five fingers of hand can all exert force and mutually hold the grip when applying force to dissect and cut the targeting object without shaking up and down. Consequently, the present invention features the effect that promotes surgical accuracy.

In accordance with a second aspect of the present invention, an effective and collaborative force transmission is provided. As a consequence, one who applies force can easily grasp the magnitude of applied force to attain easy and controllable operation and more adequate force-applying movement in dissecting and cutting a targeting object. The force transmission of the device is based on the coupling of transmission part, which enables to transmit force directly instead of through spindle and bushing, so as to directly transmit force to a two-piece sheet body at the front end and achieve the maximum benefit from direct force transmission. Moreover, the device takes the form of a horizontal grip so that five fingers of hand can all exert force and mutually hold the device in a balanced manner when applying force to dissect and cut the targeting object. Such feature makes the present invention possess the performance in adequately controlling the magnitude of applied force and operating more easily to effectively dissect and cut the targeting object. Besides, in view of less loss of force when force is outputted to the point of strength, force output can be effectively controlled and the targeting object and neighboring tissue won't be harmed due to inadequate force in surgery.

The foregoing and other features and advantages of the present invention will be more clearly understood through the following descriptions with reference to the drawing, wherein:

### BRIEF DESCRIPTION OF THE DRAWINGS

FIG. 1 is the external view showing the scissor for conventional endoscopic surgery;

FIG. 2-1 is the schematic view in accordance with the present invention, showing when the two-piece sheet body of the scissor for endoscopic surgery is in an opening state;

FIG. 2-2 is the schematic view in accordance with the present invention showing when the two-piece sheet body of the scissor for endoscopic surgery is in an closing state;

FIG. 3-1 is the first bird's view showing the combinational operation when the scissor of the present invention is in a closing state;

FIG. 3-2 is the second bird's view showing the combinational operation when the scissor of the present invention is in a closing state;

FIG. 4 is the exploded view of major parts of the scissor in accordance with the present invention;

FIG. 5-1 is the first bird's view showing the combinational operation when the scissor of the present invention is in an opening state;

FIG. 5-2 is the second bird's view showing the combinational operation when the scissor of the present invention is in an opening state;

FIG. 6 is the locally-enlarged view showing the front portion of FIG. 5; and

FIG. 7 is the locally-enlarged view showing the front portion of FIG. 3-1.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purpose of illustration and description only; it is not intended to be exhaustive or to be limited to the precise form disclosed.

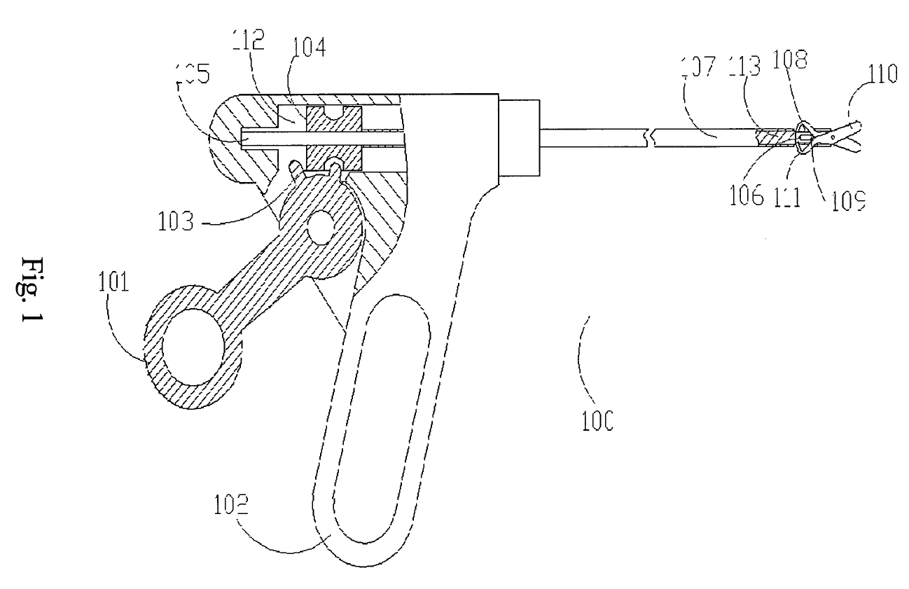

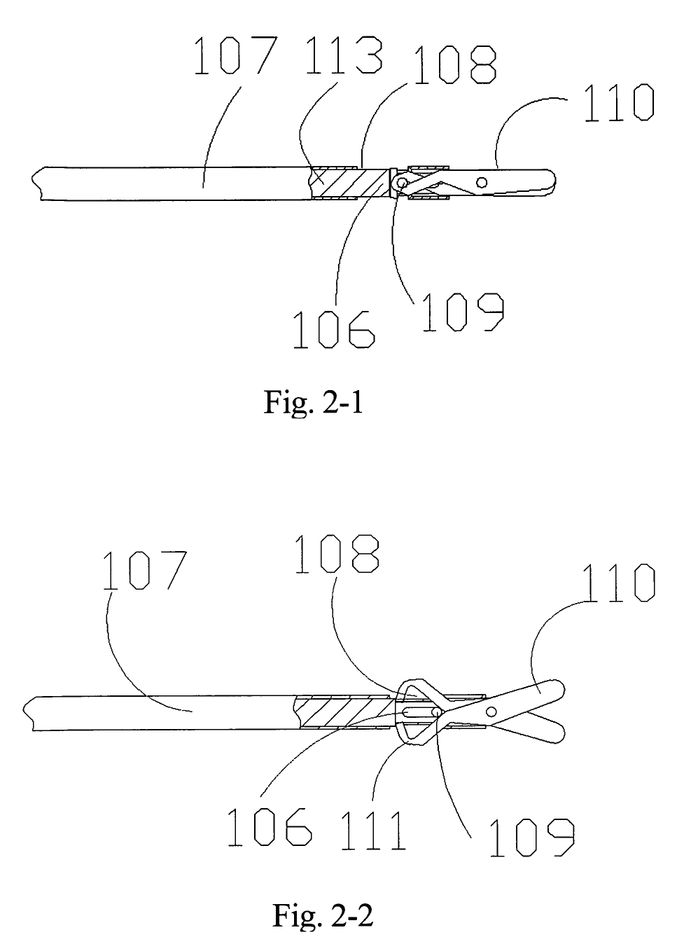

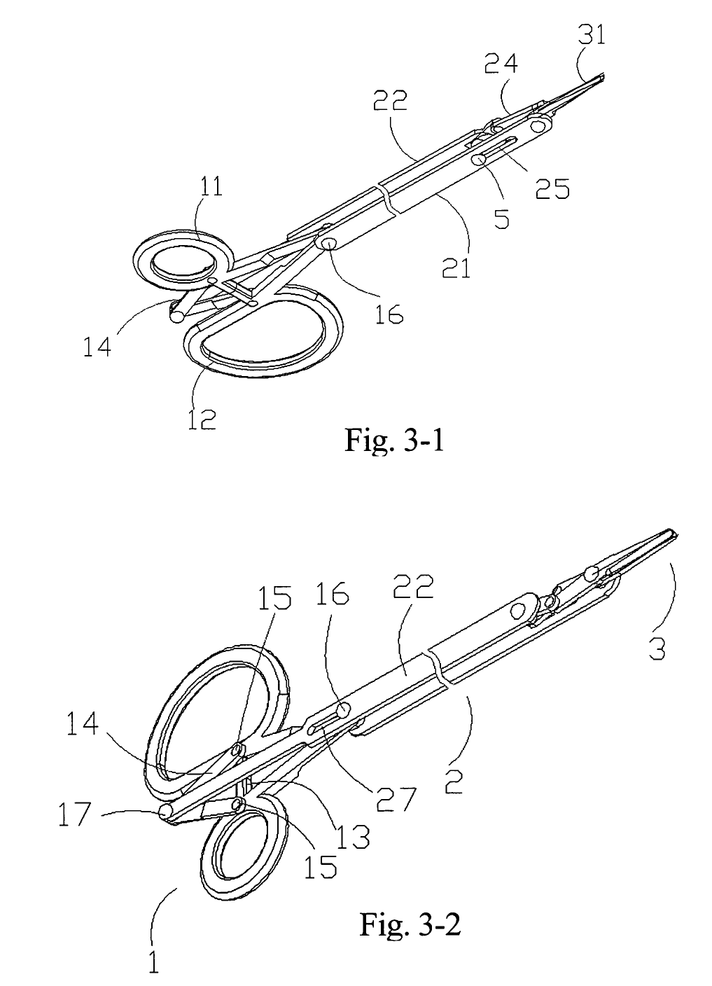

Firstly, please refer to the illustration of FIG. 3-1, FIG. 3-2 and FIG. 4, showing a device for dissecting and cutting in endoscopic surgery which mainly includes a holding part 1, a transmission part 2, and a cutting part 3. The holding part 1 contains a force application part 11, a fixed grip 12 and a grip link 14. The force application part 11 serves to let an operator to apply force to the device; the fixed grip 12 serves for the purpose of fixing while the operator is applying force. At least a stopper 13 is disposed on two corresponding inner ends of the force application part 11 and the fixed grip 12 to limit the maximum magnitude of force applied by the force application part 11. However, the stopper 13 may not be integrally built with the force application part 11 or the fixed grip 12; instead, it could be additionally formed by other material, e.g. metal, rubber or plastics. The grip link 14 is connected with the force application part 11 and the fixed grip 12 respectively to serve as a base upon applying force to activate the link. The front fastener of the grip link 15 serves for the purpose of serially connecting the grip link of the force application part and the grip link of the fixed grip. The holding part 1 can be made of metallic or non-metallic material, such as stainless steel or plastic material. The transmission part 2 is disposed at the front end of the force application part 11 and the fixed grip 12 to serve as a transmission medium for the force generated during force application and includes a fixed link 21, a transmission rod 22, and a slot 27 on the transmission rod 22. A positioning pin 16 is located at the front end of the force application part and is disposed in a rear hole 26 of the fixed link 21; the positioning pin 16 penetrates through a front hole of the fixed grip 18 to be connected with the slot of the transmission rod 27 so as to serve as the base for transmitting the force applied to the force application part 11 and the fixed grip 12. The fixed link 21 serves for the purpose of transmitting force that is stably applied. The transmission rod 22 serves as the transmission and linkage for the applied force. The starting point of the transmission rod 22 is connected with a rear fastener 17 of the grip link located at a bottom part of the grip link 14 from which the starting point extends forward to the bottom of the two-piece sheet body 31, 32. The slot 27 located on the transmission rod 27 serves as a limiting space for transmitting force applied by the force application part 11 to the positioning pin 16 at the front end of the force application part 11. The rear fastener 17 can be externally connected to a tourniquet instrument (not shown) communicated with the cutting part 3 via the transmission rod 22 for stanching blood in case of bleeding when dissecting and cutting tissue. The transmission part 2 is made of a metallic or non-metallic material and is sleeved in a rubber or plastic tube. The cutting part 3 is coupled with the front end of the transmission part 2 for the purpose of cutting off targeting object and contains a link with two-piece sheet body 24 and a two-piece sheet body 31, 32. The positioning pin of the two-piece sheet body 5 is located at the front end of the fixed link 21, is connected with the link with two-piece sheet body 24 for the two-piece sheet body 31, 32 to position while it is opened and closed, and further penetrates through the link with two-piece sheet body 24 to be connected with the transmission rod 22 and disposed in the slot 25 corresponding to the front end of the fixed link 21. The slot 25 located on the front end of the fixed link 21 serves as a limiting space for the transmission rod 22 to transmit force to the positioning pin 5. The link with two-piece sheet body 24 is disposed on the respective inner side of the fixed link 21 and the transmission rod 22 so as to drive the two-piece sheet body to open and close. Given the interlinking effect of the fastener of the two-piece sheet body 29 located at the front end of the fixed link 21 and the two-piece sheet body 31, 32, the two-piece sheet body 31, 32 is connected with the fastener for the link with two-piece sheet body 28. The two-piece sheet body 31, 32 targets at dissecting and cutting surgical portion. Depending on the surgical portion to be dissected and cut or the surgical operation to be carried out, the two-piece sheet body can be designed to have different shape, such as blunt shape, arc and feather-like teeth, and the scissor portion can also be designed to have long or short pattern.

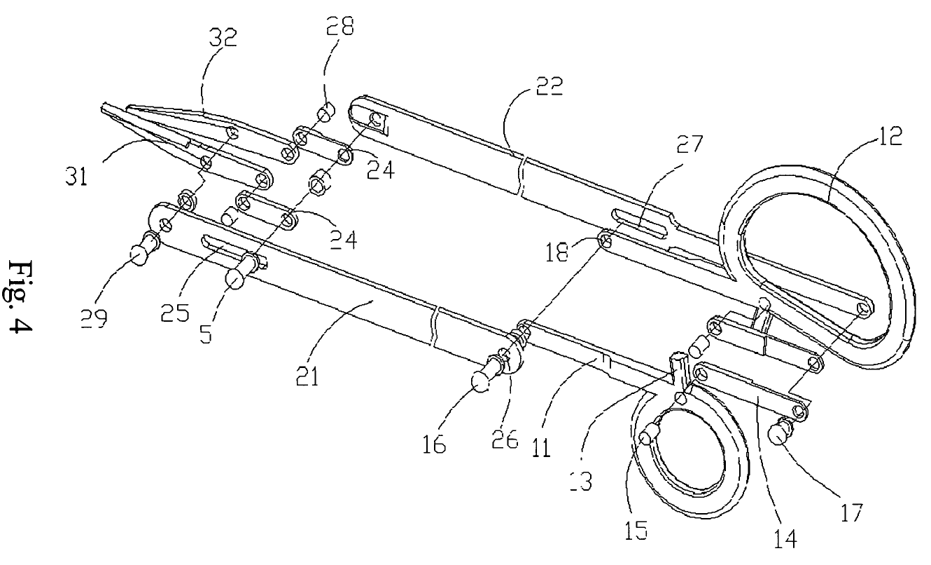

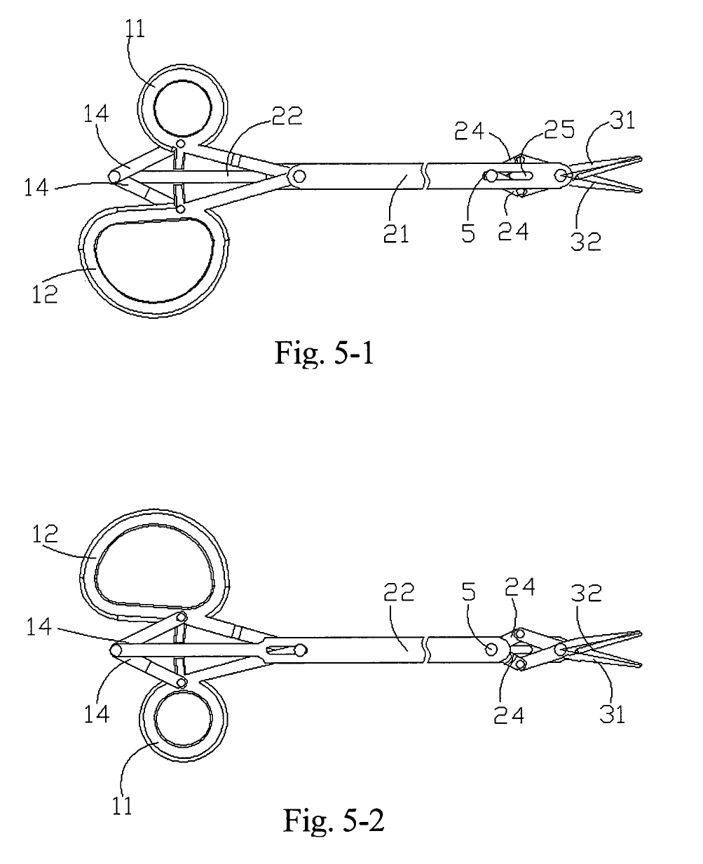

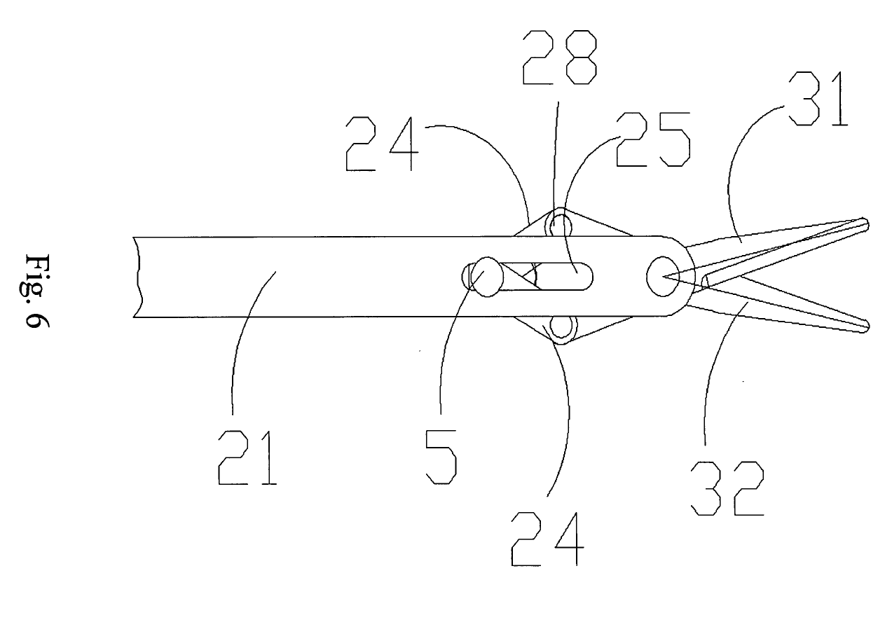

Please refer to FIG. 5-1, FIG. 5-2, FIG. 4 and FIG. 6. When a person who performs an operation applies force to the force application part 11 and holds the fixed grip 12 to push the force application part to stretch out in the left direction, the transmission rod 22 connected with the fixed grip 12 is further tugged to synchronously slide forward and the positioning pin 5 inside the slot 25 located at the front end of the fixed link 21 is driven to simultaneously move forward by the simultaneous outbound displacement of the force application part 11 and the grip link 14 connected with the fixed grip 12. The link with two-piece sheet body 24 pivoted by the positioning pin 5 is stretched out to the left and right respectively so that the left sheet body 31 and the right sheet body 32 connected with the front end of the link with two-piece sheet body 24 are simultaneously stretched out to the left and right, thus allowing the person who performs the operation to open the two-piece sheet body 24.

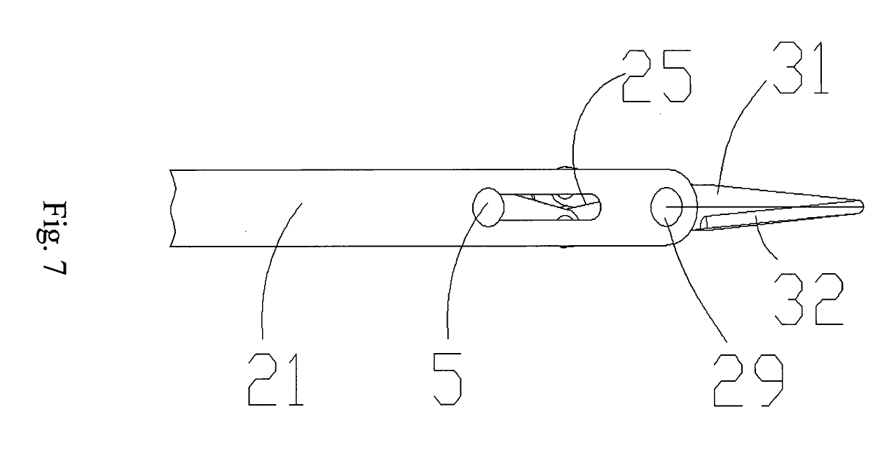

Please jointly refer to FIG. 3-1, FIG. 3-2, FIG. 4 and FIG. 7. When the person who performs the operation intends to dissect or cut a targeting object, apply force to the force application part 11 and hold the fixed grip 12 to push the force application part 11 to get close to the fixed grip 12. By synchronously sliding the force application part 11 and the transmission rod 22 connected with the fixed grip 12 backward, the positioning pin 5 inside the slot 25 located at the front end of the fixed link 21 is driven to simultaneously move backward and the link with two-piece sheet body 24 pivoted by the positioning pin 5 is synchronously driven to retract inwardly, so that the left sheet body 31 and the right sheet body 32 located at the front end of the link with two-piece sheet body 24 are folded up inwardly at the same time to complete the dissecting or cutting operation of the person who performs the operation.

In sum, the difference between the present invention and the prior art currently in use lies in:
1) The force applied by an operator, the transmission direction of force and the point of strength in the present invention are all located on the same plane and pertain to a linear relationship. Since the mechanism complies with the concept of mechanics, it is stable when applying force and its operation is the same as that of commonly used scissor. As such, the operation of conventional scissor won't be changed due to the use of endoscope, meaning that no extra work needs to be spent in learning again to make the operation more user-friendly. So far, there are designs already in use, in which their point of force application and the transmission direction of force are not co-linear, making those designs inconsistent with the operation of conventional scissor. Such inconsistency incurs the trouble of unfamiliar and awkward operation, leading to more difficulty in performing an endoscopic surgery.
2) Because the point of force application, the transmission direction of force and the point of strength are co-linear in the present invention, tissue can be dissected and cut in a more precise manner. In contrast, those of current designs already in use are perpendicular, making the force acted on the targeting point unstable and the resulting operation inaccurate.
3) In view of less loss of force outputted to the point of strength, the present invention can effectively control force output. During the operation, tissue won't be easily harmed due to improper force magnitude. The point of force application and the point of strength of conventional device form a right angle, giving rise to the difficulty in controlling the force magnitude on the point of strength in operation and less accuracy in operation.

Based on the aforementioned three points, the present invention not only is different from conventional device but also has many advantages. With the newly designed device, the endoscopic surgery will become easier and more accurate. More and more doctors are encouraged to perform such innovative endoscopic surgery so as to raise medical quality and benefit more patients from the present invention. As such, the present invention provides the practical value to the industry and the application is hereby submitted in accordance with the patent laws.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A device for dissecting and cutting in endoscopic surgery, comprising:
a holding part comprising a force application part, a fixed grip and a grip link connected with the force application part and the fixed grip respectively;
a transmission part disposed at a front end of the force application part and the fixed grip and comprising a fixed link, a transmission rod and a slot on the transmission rod; and
a cutting part connected to a front end of the transmission part and comprising a link with two-piece sheet body and a two-piece sheet body for cutting and dissecting a targeting object.

2. The device as claimed in claim 1, wherein at least a stopper is disposed on two corresponding inner ends of the force application part and the fixed grip.

3. The device as claimed in claim 2, wherein the stopper is additionally added and is made of a material selected from a group consisting of metal, rubber and plastics.

4. The device as claimed in claim 1, wherein the holding part is made of a metallic or non-metallic material selected from a group consisting of stainless steel and plastics.

5. The device as claimed in claim 1, wherein a positioning pin is disposed at a front end of the force application part.

6. The device as claimed in claim 5, wherein the positioning pin is disposed in a rear hole of the fixed link.

7. The device as claimed in claim 5, wherein the positioning pin penetrates through a hole at a front end of the holding part.

8. The device as claimed in claim 5, wherein the positioning pin is connected with the slot of the transmission rod.

9. The device as claimed in claim 1, wherein the transmission part is sleeved in a rubber or plastic tube.

10. The device as claimed in claim 1, wherein a starting point of the transmission rod is connected with a rear fastener located on a bottom part of the link with the two-piece sheet body.

11. The device as claimed in claim 1, wherein the transmission part is made of a metallic or non-metallic material.

12. The device as claimed in claim 1, wherein the slot on the transmission rod is used to form a limiting space for transmitting a force of the transmission rod to the positioning pin.

13. The device as claimed in claim 1, wherein a positioning pin of the two-piece sheet body is located at a front end of the fixed link and is connected with the two-piece sheet body.

14. The device as claimed in claim 11, wherein the positioning pin of the two-piece sheet body penetrates through the link with two-piece sheet body and is connected with the transmission rod.

15. The device as claimed in claim 11, wherein the positioning pin of the two-piece sheet body corresponds to the slot located at the front end of the fixed link and is disposed therein.

16. The device as claimed in claim 1, wherein the link with two-piece sheet body is disposed on a respective inner side of the fixed link and the transmission rod.

17. The device as claimed in claim 1, wherein the link with two-piece sheet body is connected with the two-piece sheet body by a fastener for the two-piece sheet body located at the front end of the fixed link.

18. The device as claimed in claim 1, wherein the two-piece sheet body is connected with a fastener for the link with two-piece sheet body.

19. The device as claimed in claim 1, wherein the two-piece sheet body takes the form of blunt shape, arc or feather-like teeth.

20. The device as claimed in claim 1, wherein the two-piece sheet body is designed to have a long or short length.
